(19) United States
(12) Patent Application Publication
(10) Pub. No.: US 2009/0285791 A1
(43) Pub. Date: Nov. 19, 2009

(54) Regenerating and enhancing development of muscle tissue

(75) Inventors: Antonio Giordano, Radnor, PA (US); Cristina Giacinti, Rome (IT)

Correspondence Address: REED SMITH LLP, 2500 ONE LIBERTY PLACE, 1650 MARKET STREET, PHILADELPHIA, PA, 19103, US

(73) Assignee: Sbarro Health Research Organization, Inc.

(21) Appl. No.: 12/152,558

(22) Filed: May 15, 2008

(51) Int. Cl. A61K 38/45; A61K 31/711; A61P 21/00; A61K 48/00

(52) U.S. Cl. 424/94.5; 514/44 R; 424/93.2

(57) ABSTRACT

Muscle tissue regeneration is one of the most important homeostatic processes of adult skeletal muscle, which after development retains the capacity to regenerate in response to different type of stimuli, including a direct trauma or neurological dysfunction; atrophy; and genetic defects. The present invention pertains to cdk9-55 and its ability to regenerate muscle tissue and enhance development. Cdk9-55 is specifically induced upon satellite cell differentiation and is necessary for the gene expression reprogramming required to complete the regeneration process.

### TECHNICAL FIELD OF THE INVENTION

The present invention relates to a method for regenerating muscle tissue and enhancing muscle tissue development comprising administering cdk9-55.

### BACKGROUND OF THE INVENTION

Tissue regeneration is one of the most important homeostatic processes of adult skeletal muscle, which after development retains the capacity to regenerate in response to different type of stimuli, including a direct trauma or neurological dysfunction and genetic defects (Huard et al., J. Bone Joint Surg. Am. 84-A, 822-32, 2002). The regenerative process is sustained by adult myogenic precursors, a population of quiescent mononucleated reserve cells, termed satellite cells (Charge et al., Physiol. Rev. 84, 209-238, 2004). Upon exposure to signals from the damaged environment, satellite cells are activated and start proliferating. At the molecular level, this process is characterized by the rapid up-regulation of two muscle regulatory factors (MRFs), Myf5 and MyoD. After the proliferation phase, the expression of the other two MRFs, myogenin and MRF4, favors the completion of the differentiation program. This is achieved by permanent cell cycle withdrawal, the expression of muscle-specific proteins, such as myosin heavy chain (MHC), and the fusion of myocites into the damaged fiber. A critical player in satellite cell activity is the transcription factor MyoD. Indeed, in MyoD−/− mice, there is a reduced regenerative capacity characterized by an increase in myoblast population and a decrease in regenerated myotubes (Megeney et al., Genes Dev. 10, 1173-1183, 1996); in vitro, MyoD−/− cells continue to proliferate and yield a reduced number of differentiated myocites (Sabourin et al., J. Cell Biol. 144, 631-643, 1999), indicating that MyoD plays a fundamental role in satellite cell function.

MyoD cooperates with numerous transcriptional activators and co-activators to induce the tissue-restricted expression of muscle genes (Puri et al., J. Cell. Physiol. 185, 155-173, 2000). It has been shown that cdk9 is a co-activator of MyoD and its activity is necessary for the completion of the myogenic program (Simone and Giordano, Front. Biosci. 6, D1073-D1082, 2001; Simone et al., Oncogene 21, 4137-4148, 2002; Simone and Giordano, Cell Death Differ. 14, 192-195. Erratum in: Cell Death Differ., 14, 196, 2007). Moreover, cdk9 directly interacts with MyoD in vitro (Simone et al., Oncogene 21, 4137-4148, 2002), and it takes part of a multimeric complex containing MyoD, cyclin T2a, p300, PCAF and Brg1 in muscle cells (Giacinti et al., J. Cell. Physiol. 206, 807-813, 2006). This transcriptional complex binds to the chromatin of muscle-specific genes regulatory regions to induce acetylation of specific lysines of histones H3 and H4, chromatin remodeling and phosphorylation of cdk9-specific target serines at the carboxyl-terminal domain (CTD) of RNA Polymerase II (RNApolII), and finally promote gene expression (Simone et al., Nat. Genet. 36, 738-743, 2004; Giacinti et al., J. Cell. Physiol. 206, 807-813, 2006; Simone and Giordano, Cell Death Differ. 14, 192-195. Erratum in: Cell Death Differ., 14, 196, 2007).

Recently, a second alternative isoform of the cdk9 gene has been identified in mammalian cells (Shore et al., Gene. 307, 175-182, 2003), termed cdk9-55 (molecular weight of 55 kD). This isoform originates from an additional transcription start site (TSS) located upstream to that used to generate the originally described cdk9 (now referred to as cdk9-42 due to its molecular weight of 42 kD) (Grana X., et al., Proc. Natl. Acad. Sci. USA 91, 3834-3838, 1994; Bagella et al., J Cell Physiol. 177:206-13, 1998; Bagella et al., J Cell Biochem. 78:170-8, 2000). The cdk9-55 isoform, is composed by the addition of 117 amino acids to the N-terminal domain of cdk9-42 (Shore et al., Gene. 307, 175-182, 2003). Cdk9-55 conserves all the molecular features proper of cdk9-42; in fact it associates with cyclin T, phosphorylates the CTD of RNAPolII, and its kinase activity is specifically inhibited by 5,6-dichloro-1-β-d-ribofuranosylbenzimidazole (DRB) (Shore et al., Gene. 307, 175-182, 2003; Liu and Herman, J. Cell. Physiol. 203, 251-260, 2005).

The relative abundance of the two isoforms varies across different tissues: cdk9-55 is predominantly expressed in lung, liver and brain, whereas cdk9-42 predominates in spleen and testis (Shore et al., Gene. 307, 175-182, 2003; Shore et al., Gene 350, 51-58, 2005). HeLa human cervical carcinoma cells and NIH3T3 mouse fibroblasts express higher levels of cdk9-42 protein, than the cdk9-55 isoform (Shore et al., Gene. 307, 175-182, 2003). Moreover, in primary undifferentiated monocytes, cdk9-55 expression is not detected although cdk9-42 is present at high levels; however, cdk9-55 expression is induced upon macrophage differentiation (Liu and Herman, J. Cell. Physiol. 203, 251-260, 2005). When macrophages are stimulated with LPS or infected with HIV, the ratio between the two isoforms is reversed, since cdk9-42 becomes the predominant form (Shore et al., 307, 175-182, 2003). Activation of primary lymphocytes increases the levels of cdk9-42, while the levels of cdk9-55 decrease or remain steady following activation (Liu and Herman, J. Cell. Physiol. 203, 251-260, 2005). Finally, rat hepatocytes express more cdk9-55 than cdk9-42, but in primary culture they exhibit increased cdk9-42 levels while those of cdk9-55 stay relatively constant over time (Shore et al., Gene 350, 51-58, 2005). Interestingly, subcellular localization studies indicated that cdk9-55 is exclusively expressed in the nucleus (Shore et al., Gene 350, 51-58, 2005), while cdk9-42 can occupy both the cytoplasm and nucleus (De Falco et al., Oncogene 21, 7464-7470, 2002).

These findings indicate that the expression of the two isoforms is differentially regulated in a signal-dependent and cell type-specific manner, and suggest that cdk9-55 could represent the isoform involved in the differentiation program of different tissues. It has been reported that cdk9 transactivates PPARγ-mediated gene expression and promotes adipogenesis (Iankova et al., Mol. Endocrinol. 20, 1494-1505, 2006). In these cells, cdk9-55 is strongly up-regulated at both the mRNA and protein levels upon the induction of the differentiation process.

The capacity of muscle tissue to regenerate in response to injury represents an important homeostatic process that is impaired with age or in pathological conditions of the musculature like injury (Corsi et al., Current Genomics 5, 7-17, 2004; Jarvinen et al., Research in Clinical Rheumatology 21, 317-331, 2007), genetic (muscular dystrophies) (Deconinck et al., Pediatric Neurology 36, 1-7, 2007; Radley et al., International Journal of Biochemistry & Cell Biology 39, 469-477, 2007) or chronic diseases (ranging from cancer to AIDS, from chronic heart failure to kidney disease) (Musaro et al., Cell Transplantation 15, S128, 2006). The diminished muscle regeneration is due to exhaustion over time of satellite cells in muscular dystrophies (Deconinck et al., Pediatric Neurology 36, 1-7, 2007), to inability of activation (as in old muscle tissue) (Conboy et al., Cell Cycle 4, 407-410, 2005) or decrease in differentiative potential (as in chronic disease) (Tisdale, Nature Reviews Cancer 2, 862-871, 2002) of satellite cells that respond to an altered environment. Many different therapeutic approaches have been developed, giving rise to an increase in impaired muscle regenerative mechanisms but without completely rescuing the altered phenotype. To this purpose many different strategies have been performed: transplant of healthy satellite cells (Gussoni et al., Nature Medicine 3, 970-977, 1997) or bone marrow (BM) stem cells (Goodell, Biotechniques 35, 1232, 2003; Gussoni, Nature Medicine 3, 970-977, 1997), gene therapy by encapsidated adenovirus minichromosomes or adeno-associated viral vectors (Chamberlain et al., Neuromuscular Disorders 15, 741, 2005; Gregorevic et al., Journal of Gene Medicine 9, 529, 2007) uses of anabolic steroids (Balagopal et al., Journal of Physiology-Endocrinology and Metabolism 290, E530-E539, 2006), growth factors such as IGF- (Espinoza-Derout et al., Cardiovascular Research 75, 129-138, 2007; Musaro et al., Nature Genetics 27, 195-200, 2001) or anti-inflammatory drugs (NSAID) and glucocorticoids used specifically in the treatment of muscle injures in humans (O'Grady et al., Medicine and Science in Sports and Exercise 32, 1191-1196, 2000). Recently, it has been discovered that mesoangioblasts (De Angelis et al., Journal of Cell Biology 147, 869-877, 1999) isolated from diagnostic muscle biopsies of Inflammatory myopathies (IM) fail to differentiate into skeletal myotubes (Morosetti et al., Proceedings of the National Academy of Sciences of the United States of America 103, 16995-17000, 2006); a myogenic inhibitory basic helix-loop-helix factor B3 is highly expressed in inclusion-body myositis (IBM) mesoangioblasts. Silencing this gene or over-expressing MyoD rescues the myogenic defect of IBM mesoangioblasts. Chronic diseases like cancer and AIDS induce muscle cachexia and impair muscle regeneration (Musaro et al., Nature Genetics 27, 195-200, 2001); inhibitors like TNF-α (Barton et al., Journal of Cell Biology 157, 137-147, 2002) downregulate the myogenic factors MyoD and myogenin, blocking differentiative muscle pathways (Guttridge et al., Science 289, 2363-2366, 2000; Szalay et al., European Journal of Cell Biology 74, 391-398, 1997).

Cdk9-55 has been well characterized, but up until now, its biological activity has been uncertain and the mechanism by which it acts has been unknown and subject to ongoing research (Shore et al., Gene 350, 51-58, 2005).

### SUMMARY OF THE INVENTION

The present invention pertains to a method for regenerating muscle tissue and enhancing development of muscle tissue, comprising administering a vector encoding cdk9-55 to a patient in need thereof. The vector may be an adenoviral vector or a retroviral vector. The vector may be administered intra-arterially, intravenously, or intramuscularly. The muscle tissue may be smooth muscle, cardiac muscle, and/or skeletal muscle. The present invention also pertains to a method for regenerating muscle tissue or enhancing development of muscle tissue, comprising co-administering a vector encoding cdk9-55 and Cyt2a, MyoD, or at least one muscle regulatory factor to the patient in need of muscle tissue regeneration. The present invention also pertains to a method for regenerating muscle tissue or enhancing development of muscle tissue, comprising administering to a patient a composition comprising cdk9-55 proteins. Further, at least one muscle regulatory factor and/or adjuvant may be administered with cdk9-55.

### BRIEF DESCRIPTION OF THE DRAWINGS

The present invention is best understood with reference to the following detailed description of the invention and the drawings in which:

FIG. 1A shows an immunoblot analysis of total cells extracts of cdk9 isoforms, myogenin and MHC;

FIG. 1B shows the expression of cdk9 isoforms, RNApolII, and the phosphorylation of CTD-serine 2 that were monitored by immunoblot (DM=24 h);

FIG. 1C shows the quantitative analysis performed to evaluate the expression of cdk9-55, myogenin and MCK (DM=24 h);

FIG. 1D shows that differentiating C2C12 were exposed (+) or not (−) to DRB 100 μM;

FIG. 1E shows the immunoblot performed with the indicated antibodies;

FIGS. 2A and 2B show immunofluorescence studies demonstrating the co-expression of cdk9 with MyoD and Desmin during the activation phase;

FIG. 2C shows immunofluorescence studies demonstrating the co-expression of cdk9 with MHC during differentiation;

FIGS. 2D and 2E shows cdk9 isoforms were detected by immunoblot (IB) in cell extracts and MyoD immunoprecipitations (IP);

FIG. 2F shows cdk9-55 and MCK expression levels determined by quantitative RT-PCR;

FIG. 3A shows frozen sections of regenerating fibers stained with anti-cdk9 and Hoecst (cardiotoxin (CTX)=48 h);

FIG. 3B shows protein extracts directly probed;

FIG. 3C shows protein extracts first subjected to immunoprecipitation (IP) with anti-MyoD with the indicated antibodies (CTX=48 h);

FIG. 3D shows quantitative analysis of cdk9-55, Desmin and neonatal-MHC (neo-MHC) gene products in uncrushed (Cntr) or CTX injected muscles (CTX=48 h);

FIGS. 3E and 3F shows time course experiments monitoring protein and mRNA levels of cdk9-55 during muscle regeneration.

FIG. 4A shows cdk9DN or the empty vector (pcDNA3.1) was electropored into injured muscle and regeneration was monitored by immunoblot;

FIG. 4B shows cdk9DN or the empty vector (pcDNA3.1) was electropored into injured muscle and regeneration was monitored by Real-Time PCR;

FIG. 4C shows an evaluation of the transfection efficiency using a GFP reporter and the GFP-positive myofibers counted; and

FIG. 4D shows the reduction in the number of β-GAL-positive fibers when cdk9DN was over-expressed.

### DETAILED DESCRIPTION OF PREFERRED EMBODIMENTS

Reference will now be made in detail to embodiments of the invention. While the invention will be described in conjunction with the embodiments, it will be understood that they are not intended to limit the invention to those embodiments. On the contrary, the invention is intended to cover alternatives, modifications, and equivalents, which may be included within the spirit and scope of the invention as defined by the appended claims.

The present invention pertains to the regeneration of muscle tissue in a patient by administering a vector encoding cdk9-55 or a protein encoded by the cdk9-55 gene. The term “cdk9-55” as used herein refers to the gene, the protein expressed by the gene, and/or the gene in a vector or plasmid. The term “muscle tissue” as used herein refers to any muscle tissue of the body, including, but not limited to smooth muscle, cardiac muscle, and skeletal muscle (striated muscle).

The present invention is applicable to many types of muscle tissue injury and disease, including, but not limited to mechanical injury, such as, but not limited to acute and chronic strains; loss of muscle tissue due to disease or injury; cardiac muscle-cell hypertrophy; atrophy; genetic disorders such as, but not limited to muscular dystrophies; chronic disorders such as, but not limited to AIDS, cancer, chronic heart failure, and kidney disease; and diseases related to aging.

The present invention may be administered by any mechanism known in the art. Cdk9-55 may be administered directly to muscle tissue or systemically. Cdk9-55 may be administered directly to the muscle by injection of the protein or through in vivo naked plasmid DNA electrotransfer and adenoviral injection. Cdk9-55 may also be administered systemically through hydrodynamic gene delivery of adeno-associated viral vectors into a vein or artery of a human. (Chamberlain et al., Neuromuscular Disorders 15, 741, 2005; Gonin et al., Journal of Gene Medicine 7, 782-791, 2005; Herweijer et al., Gene Therapy 14, 99-107, 2007). The systemic administration may be through the use of a viral vector encoding cdk9-55 or the protein encoded by the ckd9-55 gene. The vector or protein may be packaged in any form known in the art for systemic delivery. The viral vector may be an adenoviral vector or a retroviral vector, preferably an adenoviral vector. Cdk9-55 may also be administered systemically through a vein or artery, preferably the femoral artery.

According to an embodiment, cdk9-55 may be impregnated or coated on a resorbable material and applied to the injured or diseased muscle tissue, or area missing muscle tissue. Cdk9-55 may also be incorporated with a slow release implant as known in the art. Cdk9-55 may also be applied to healthy muscle tissue to generate additional muscle tissue. Any other conventional delivery techniques known in the art are envisioned for administering cdk9-55.

Cdk9-55 may be administered using a pharmaceutically acceptable carrier known in the art.

The nucleotide sequence of cdk9-55 (SEQ ID NO: 1 and SEQ ID NO:3) may be incorporated into a plasmid or vector.

The protein sequence of cdk9-55 (SEQ ID NO:2 and SEQ ID NO:4) may be synthesized and/or purified, and administered by techniques well known in the art. Substitution of equivalent amino acids (i.e. conservative substitutions) in SEQ ID NO:2 or SEQ ID NO: 4 would not be expected to affect the cdk9-55 protein's activity. These amino acid substitutions would be envisioned by those of ordinary skill in the art. Such equivalent amino acid sequences are also included within the present invention.

Cdk9-55 may also be used in combination with cdk9-42 (SEQ ID NO:5, SEQ ID NO:6, SEQ ID NO:7, and SEQ ID NO:8) and/or any other muscle regulatory factors (MRF). Cdk9-55 may also be used in combination with an adjuvant, drug, or treatment that may affect muscle tissue regeneration and enhancing the development of existing muscle tissue. According to an embodiment, cdk9-55 is administered in combination with Cyt2a. Cyt2a is the cyclin protein that regulates cdk9 activity (Simone et al., Nat. Genet. 36, 738-743, 2004; Giacinti et al., Journal of Cellular Physiology 206, 807-813, 2006) It has been shown that the complex ckd9-(42/55)/Cyt2a acts with the MRF-Myod in modulating muscle gene transcription both in vitro and in vivo (Giacinti et al., Journal of Cellular Physiology 206, 807-813, 2006; Simone et al., Oncogene 21, 4137-4148, 2002). Cdk9-55 (Giacinti et al., Journal of Cellular Physiology 206, 807-813, 2006; Simone et al., Frontiers in Bioscience 6, D1074-D1082, 2001; Simone et al., Oncogene 21, 4137-4148, 2002) may also be complexed with the myogenic factor MyoD to modulate muscle differentiative pathways.

According to an embodiment, healthy, injured, and/or diseased muscle tissue may be taken from a human or animal to generate more muscle tissue through the administration of cdk9-55 or a combination of cdk9-55 and at least one MRF, adjuvant, and/or drug. Following regeneration, the muscle tissue can be transplanted back to the injured or diseased area.

### Delivery of DNA:

The constructs of cdk9-55, such as pcDNA3-cdk955Tag can be made by techniques well-known in the art. (Simone et al., Oncogene 21, 41337-4148, 2002; Bloquel et al., Journal of Gene Medicine 6, S11-S23, 2004). Cdk9-55-adeno-associated viral vectors (e.g. rAAV-serotype 6, -serotype 8, or -serotype 9) can also be made by techniques well-known in the art (Salva et al., Molecular Therapy 15, 320-329, 2007).

The following techniques may be used to carry out the present invention, but the present invention is not limited to these techniques and other techniques known in the art may be used:

1. In Vivo Naked Plasmid DNA Electrotransfer

Naked DNA can be injected directly into the muscle by the electrotransfer method (Dona et al., Biochemical and Biophysical Research Communications 312, 1132-1138, 2003; Trollet et al., Current Gene Thereapy 6, 561-578, 2006). In vivo electrotransfer is a physical method of gene delivery in various tissues (including muscle) and organs, relying on the injection of a plasmid DNA followed by electric pulse delivery. Briefly, DNA (approximately 0.06-approximately 25 mg) in about 50 ml of 0.9% NaCl is injected with a syringe in a proximal to distal direction. Then, a pair of spatula-like electrodes (e.g. 0.5 cm wide, 2 cm long) is placed at each side of the muscle and electric pulses are delivered. For example, approximately five electric pulses with a fixed pulse duration of approximately 15 ms to 20 ms and an interval of approximately 180 ms to 200 ms are delivered using an electric pulse generator (Electro Square porator ECM 830, BTX, San Diego, Calif.). The ratio of applied voltage to electrode distance of approximately 18 V/cm to 30 V/cm.

2. Naked DNA or rAAV Hydrodynamic Delivery:

For naked DNA hydrodynamic gene delivery, plasmids are purified from bacterial culture using an Endofree Mega kit (Qiagen). For example, small amounts of DNA are diluted in about 1.6 ml of sterilized 0.9% NaCl solution and injected into a vein or artery, using a needle. The needle may be of any gauge necessary for the procedure, e.g. 27.5-gauge needle. Gregorevic et al., Molecular Therapy 9, S274, 2004; Sebestyen et al., Journal of Cell Science 108, 3029-3037, 1995.

For recombinant adeno-associated viruses (rAAV), the rAAV is administered via a vein or artery. For example, in mice, 3-4×10¹² genome copies of rAAV vector are administered via the tail vein.

For employing mouse cdk9-55, cDNA for cdk9-55 is subcloned into an expression plasmid in which the transgene is driven via a specific muscle promoter, such as but not limited to desmin or MLC, to ensure specific expression of the gene in the muscle compartment (Chamberlain et al., Neuromuscular Disorders 15, 741, 2005).

For rAAV vectors, any vector known in the art can be used, including but not limited to rAAV-serotype 6, -serotype 8, or -serotype 9 (Gregorevic et al., Journal of Gene Medicine 9, 529, 2007). The design for tissue-specific regulatory cassettes for high-level rAAV-mediated expression in muscle tissue, such as smooth, skeletal, and cardiac muscle, can be used (Salva et al., Molecular Therapy 15, 320-329, 2007).

### Purification of Proteins:

A protein and/or polypeptide (collectively referred to herein as “protein(s)”) of the present invention pertains to a protein that is free of cellular components and/or contaminants normally associated with a native in vivo environment. The proteins used in the present invention include any isolated naturally occurring allelic variant, as well as recombinant forms thereof. The proteins of the present invention can be isolated, synthesized, and purified using various methods well-known to those of skill in the art (Shore et al., 307, 175-182, 2003 and Shore et al., Gene 350, 51-58, 2005). The methods available for the isolation and purification of proteins include precipitation, gel filtration, ion-exchange, reverse-phase and affinity chromatography, and the like. Other well-known methods are described in Deutscher et al., Guide to Protein Purification: Methods in Enzymology Vol. 182, (Academic Press, (1990)) or Roy, et al., J Chromatogr B Analyt Technol Biomed Life Sci. 849(1-2), 32-42, 2007, which are each incorporated herein by reference in its entirety. Alternatively, the proteins can be obtained using well-known recombinant methods as described, for example, in Sambrook et al., Molecular Cloning: A Laboratory Manual 2d Ed. (Cold Spring Harbor Laboratory, (1989); which is incorporated herein by reference in its entirety.

The following is a method of protein purification that may be used with the present invention.

Preculture (20 mL) of one single colony E. coli (DE3) pLys containing the recombinant plasmid cdk9-55 will be diluted in 500 mL of Luria-Bertani medium (LB) supplemented with appropriate antibiotics (carbenicillin (50 gm/L) and chloramphenicol (50 gm/L)). The culture will be conducted at 30° C. at 180 rpm in a shaking incubator until the cells reached mid-log growth (OD600 measurements of 0.4-0.6). At this point, the expression of the target protein is induced by adding IPTG (0.1 mM) and continued incubation at 30° C. for 3 h. The cells will be harvested by centrifugation and resuspended in 30 mL lysis buffer (10 mM Tris-HCl, pH 7.4, 25 mM NaCl, 1 mM EDTA, and 1 mM PMSF). After sonication (three short bursts, about 30 s each, allowing the bacterial suspension to cool on ice between each burst), the lysate will be clarified by centrifugation for 1 h at 20,800 g and 4° C. The supernatant will be loaded over a DEAE Sepharose column pre-equilibrated with lysis buffer. The absorbed proteins will be eluted with a linear gradient (25-500 mM NaCl, elution volume 60 mL) by the use of a peristaltic pump at 60 mL/h. After, the fractions will be visualized in silver stained SDS gels, the selected CDK9-55 fractions were pooled and dialyzed against a buffer containing 10 mM Hepes, pH 7.4, 25 mM NaCl, and 1 mM EDTA. The dialyzed CDK9 will be loaded onto an ATP affinity column pre-equilibrated with buffer A (10 mM Hepes, pH 7.4, 25 mM NaCl, 1 mM EDTA, 10% glycerol (v/v), and 0.5 mM dithiothreitol). After washing, bound proteins will be eluted with a 50 mL linear salt gradient (25-500 mM NaCl in buffer A). Fractions containing CDK9-55 will be pooled and concentrated (up to approximately 6 mg/mL) and dialyzed using an Amicon ultrafiltration cell (MWC 10,000 Da) against 10 mM Hepes, pH 7.4, and 1 mM EDTA. The protein content will be analyzed by SDS-PAGE and visualized using the Silver Staining kit, Protein (GE HealthCare).

All of the previous studies on cdk9 were carried out with kinases that were immunoprecipitated from cell extracts or by epitope-tagged recombinant kinases that were purified by analytical scale affinity schemes.

In many cases, the homogeneity of the preparations and the presence of contaminating kinase activities were not systematically addressed. This concern is especially valid when working with immunoprecipitated complexes, which may contain other kinases that are difficult to track. If the immunoprecipitates are derived from crude extracts, it is also possible that the experiment will be conducted with multiple forms of these complexes that share the same CDK component.

In order to circumvent these problems and the variance that could result from the differences in the purification strategies, we propose to use a uniform procedure for the purification of recombinant CDK9-55 complexes. Using these recombinant kinases, we identified additional differences in the preference of these kinases towards different parts of the pol II CTD. This procedure generates large amounts of kinases that are practically devoid of other kinase activities. This procedure enabled us to identify distinct preferences of CDK9 towards different parts of pol II CTD. The CTD consists of 52 repeats of a Ser/Thr-rich consensus heptapeptide which might attract promiscuous kinase activities.

Another purification method that may be used with the present invention is as follows:

Expression vectors, such as baculovirus expressing His6-CDK9-55/CycT2 may be used. Recombinant baculovirus for the expression of His6-CDK9/CycT1 will be produced using the Novagen Baculovirus expression system. This system utilizes a Bacvector-3000 (Novagen) triple cut virus DNA, which is a modified form of the AcNPV genome. A transfer plasmid cassettes for the expression of His6-CDK9-55 and CycT2 (pBAC-CDK9-55/CycT2), and Bacvector-3000 DNA were co-transfected into Sf9 cells. The recombinant baculovirus will be amplified and used for protein expression.

Amplification of recombinant viruses High titer viral stocks for the production of recombinant proteins will be prepared from low passage (1 or 2) viral stocks by a two step amplification procedure. Sf9 cells at density of 0.1-0.3×106 cells/ml will be infected with individual viruses at a multiplicity of infection (MOI) of 0.1-1. The infected cells will be incubated for 5-6 days. The efficiency of infection will be monitored by the loss of adherence and the larger size of the cells. The supernatant from these cultures will be used to infect a larger Sf9 culture of density 0.5-1×106 cells/ml at a MOI of 1. The culture was incubated for 4-5 days, cells were spun and the supernatant will be immediately used or stored at −80° C. for up to six months. This procedure typically produces viral stocks of ˜1×108 pfu/ml.

### Expression of Recombinant Kinases:

Expression of recombinant CDK complexes will be conducted by co-infecting about 1.5-2×109 Sf9 cells (1.5-2×106 cells/ml) with the appropriate combination of baculoviruses at MOI 4 for each individual virus. The cells will be harvested after 48 hours by spinning at 275 g for 5 minutes at 4° C., washed with PBS and frozen (−80° C.) in 15 ml of lysis buffer (10 mM Tris.HCl pH 7.5, 10 mM NaCl, 2 mM β-mercaptoethanol, 0.5 mM EDTA, 10 mM 2-glycerophosphate, 0.5 mM Na-vanadate, 2 mM NaF, 2 μg/ml leupeptin, 2 μg/ml aprotonin, 2 μg/ml pepstatin, 0.2% (v/v) NP-40, 50 μg/ml PMSF). Alternatively, the cells will be immediately lysed in lysis buffer by 10 strokes with a Dounce homogenizer. After cell lysis, the proteins will be extracted by adding 0.5M NaCl and 5 mM imidazole, and rocking for 30 min at 4° C. The extract will be clarified by spinning in a SW50.1 rotor (Beckman) at 75000 g for 30 min and immediately processed by metal (Ni2+) affinity chromatography. Ni2+-NTA pull-down assay. Pull down assays will be performed with 250 μl aliquots of Sf9 cell extracts and 50 μl of 50% (v/v) Ni2+-NTA agarose beads equilibrated with 10 mM Tris.HCl, pH 7.6, 0.5 M NaCl, 5 mM imidazole, 50 μg/ml PMSF and 10% (v/v) glycerol (buffer A).

The suspension was rocked on a nutator for 1 h and the beads will be pelleted by spinning for 1 minute at 3000 rpm. The beads will be then washed five times with 1 ml of buffer A+0.1 M NaCl, boiled for 5 minutes in SDS-sample buffer and further analyzed by Western blot or silver staining.

### Purification of CDK Complexes by Ni2+-NTA Chromatography:

The cell extract from about 1 liter of infected cells will be mixed with 1 ml of Ni2+-NTA agarose beads (Qiagen) that will be equilibrated with 10 mM Tris.HCl pH 7.6, 0.5 M NaCl, 5 mM imidazole, 50 μg/ml PMSF and 10% (v/v) glycerol, and rocked on a Nutator for 1 h. The beads will be washed once in the equilibration buffer and transferred to a disposable 10 ml column (Amersham). Bound proteins will be step-wise eluted with 15, 25, 100 and 400 mM imidazole in 10 mM Tris-HCl pH 7.6, 0.1 M NaCl, 50 μg/ml PMSF and 10% (v/v) glycerol. The fractions containing the recombinant protein kinases will be identified by SDS-PAGE/Coomassie Brilliant Blue R-250 staining, pooled and stored at −80° C.

### Purification of CDK Complexes by Mono S Chromatography:

The pooled protein fractions from Ni2+-NTA chromatography will be buffer exchanged in PD10 columns (Amersham) to 25 mM HEPES pH 7.6, 0.1 mM EDTA, 1 mM DTT, 5% (v/v) glycerol, 50 μg/ml PMSF and 80 mM NaCl. The proteins will be loaded on a tandem of two 5 ml Econo-Pac Mono S cartridges (BioRad) and eluted with a linear 0.08-0.5M NaCl gradient in 25 mM] HEPES pH 7.6, 0.1 mM EDTA, 1 mM DTT, 50 μg/ml PMSF, and 5% (v/v) glycerol. The fractions containing recombinant protein kinases will be identified by SDS-PAGE/silver staining and stored at −80° C.

### Kinase Assays:

Kinase substrates: Glutathione-S-transferase carboxyl terminal domain (GST-CTD).

The kinase assays will be performed in a volume of 20 μl containing 20 mM Tris.HCl, pH 8, 50 mM KCl, 7 mM MgCl2, 5 mM 2-glycerophosphate, 100 μg/ml BSA (2 μg), 10 μM ATP, 2 μCi (7.4×104 Bq) α-32P-ATP (ICN), 40 μg/ml (800 ng) GST-CTD or maltose-binding proteing (MBP) and about 100-400 ng/ml (2-8 ng) of purified kinase. These amounts correspond to 100-500 fold molar excess of substrate versus kinase. It is important to note that the GST-CTD molecule has at least 52 sites of phosphorylation (52 repeats with a consensus YSPTSPS (SEQ ID NO:9) on a single molecule, thus additionally increasing the kinase/substrate ratio. MBP also contains multiple sites of phosphorylation. Under the described conditions the kinase reactions are linear for at least three hours (data not shown). The kinase reactions were incubated for 30 minutes at 30° C. and terminated by the addition of SDS-PAGE loading buffer and then boiled for 5 minutes. Aliquots will be analyzed by SDS-PAGE gels and autoradiography. The incorporation of ATP in GST-CTD (1-52) and MBP (pmol of ATP/min/mg of protein) will be determined. Kinase assays will be also performed in the presence of kinase inhibitors, DRB (5,6-dichlorobenzimidazole riboside), and roscovitine (2-(1-ethyl-2-hydroxyethylamino)-6-benzylamino-9-isopropylpurine). DRB will be dissolved at 50 mM in 95% ethanol and stored at −20° C. Working dilutions of 800, 200 and 40 μM in water will be prepared at the time of assay and immediately added to the kinase reaction. Roscovitine will be dissolved in DMSO at 50 mM and stored at −20° C. Dilutions in water were made prior to the reactions and immediately added to the kinase reaction.

In order to determine the level of regeneration in a muscle tissue or the enhancement of development of existing muscle tissue, regenerative molecular markers are analyzed both at protein and RNA levels by Western blot and Real-Time PCR assays, respectively. Protein muscle extraction is performed by muscle homogenization in modified lysis buffer (10 mM Tris HCl, pH 7.4, 150 mM NaCl, 1% Nonidet P-40, 1% sodium deoxycholate, 0.1% SDS, 10% glycerol) (Giacinti et al., Journal of Cellular Physiology 206, 807-813, 2006).

### Delivery of Protein:

Cdk9-55 may be delivered by any mechanism known in the art, including, but not limited to impregnating or coating cdk9-55 on a resorbable material; incorporating cdk9-55 into a slow release implant; and directly administering the cdk9-55 protein using a pharmaceutically acceptable carrier to the muscle tissue, e.g. injection. These techniques of delivering a cdk9-55 protein may be applied to injured muscle tissue, diseased muscle tissue, areas of missing muscle tissue, e.g. due to surgery or injury, and/or atrophied muscle tissue, e.g. due to non-use.

Cdk9-55 may also be delivered using SAINT-PhD (Synvolux Therapeutics B.V., Groningen, Netherlands). SAINT-PhD consists of a cationic pyridinium amphiphile and helper lipid. Upon mixture of SAINT-PhD with cdk9-55 protein, a particle of approximately 200 nm in diameter is formed. In this particle, the cdk9-55 protein is enwrapped by at least one bilayer of lipids. Furthermore, in the complex formed only non-covalent interactions are present between SAINT-PhD and the cdk9-55 protein. The cationic amphiphiles on the surface of the particle have high affinity for the negatively charged cell surface. Upon fusion or entrapment of the particle, the protein is released into the cytoplasm of the cell. The proteins delivered by SAINT-PhD are functional and modified. The cdk9-55 and SAINT-PhD complex may be injected directed into the muscle tissue.

The following examples are intended to illustrate, but not to limit, the scope of the invention. It is to be understood that other procedures known to those skilled in the art may alternatively be used.

### EXAMPLE 1

### Cell Culture

C2C12 cells were grown in DMEM supplemented with 20% FBS (GM) or with 2% HS (DM).

Single muscle fibers with associated satellite cells were isolated as described in Rosenblatt et al., In Vitro Cell. Dev. Biol. Anim. 31, 773-779, 1995. Briefly, the hind limb muscles were digested for 60 minutes at 37° C. in 0.2% collagenase I (Sigma-Aldrich) and triturated with a wide-bore pipette. Single myofibers were then cultured in 6-well plates (10 fibers/well) pre-coated with ECM gel (Sigma-Aldrich). Fiber cultures were grown in DMEM supplemented with 10% HS and 0.5% chick embryo extract (MP Biomedicals, Solon, Ohio). Three days later, the fibers were removed, and proliferation of detached cells was induced by DMEM supplemented with 20% FBS, 10% HS, and 1% chick embryo extract. After 4-5 days, the cells were allowed to differentiate by DMEM with 2% HS and 0.5% chick embryo extract.

### EXAMPLE 2

### Muscle Regeneration and Immufluorescence Studies

Quadriceps and tibialis muscles from C57BL6J mice and 22-month Desmin/nls-LacZ mice were injected with 30 μl of 10 μM cardiotoxin. Mice were anesthetized before cervical dislocation and muscle tissue was separated from bone and most connective tissue and immediately frozen in liquid nitrogen. Frozen sections were fixed in 4% paraformaldehyde 10 min on ice, and then stained with X-gal or pre-incubated in PBS containing 1% BSA, 1:100 goat serum for 1 h at RT, and processed as described in Musarò et al., Nature Genetics 27, 195-200, 2001. Antibodies specific for MyoD, cdk9, Desmin and MHC were used. Nuclei were visualized using Hoechst staining.

### EXAMPLE 3

### Plasmids and In Vivo Muscle Transfection by Electric Field

The construct pcDNA3-cdk9DN-HATag expressing dominant negative cdk9 has been described (Simone et al., Oncogene 21, 4137-4148, 2002). In vivo experiments were carried out as described in Dona et al., Biochemical and Biophysical Research Communications 312, 1132-1138, 2003. Briefly, injured quadriceps and tibialis muscles from C57BL6J mice and 22-month Desmin/nls-LacZ mice were exposed by a short incision and DNA (0.06-25 mg) in 50 ml of 0.9% NaCl was injected with a Hamilton syringe in a proximal to distal direction. Then, a pair of spatula-like electrodes (0.5 cm wide, 2 cm long) were placed at each side of the muscle and electric pulses were delivered. Five electric pulses with a fixed pulse duration of 20 ms and an interval of 200 ms were delivered using an electric pulse generator (Electro Square porator ECM 830, BTX, San Diego, Calif.). The ratio of applied voltage to electrode distance was 50 V/cm.

### EXAMPLE 4

### Immunoblotting and Immunoprecipitation

Immunoprecipitations were performed with anti-MyoD antibody (Santa Cruz, Calif.) followed by the addition of protein A-sepharose. Beads were extensively washed and loading buffer without β-mercaptoethanol was added at 4° C. to work in non-denaturing conditions. Samples were resolved in PAA-Gels and transferred to a Hybond-ECL nitrocellulose (Amersham, IL). The blots were blocked with TBST containing 5% non-fat dry milk. Antibodies specific for cdk9, myogenin, MHC, RNAPolII, P-Serine2-CTD, Desmin and tubulin were used as described in Simone et al., 2002; Giacinti et al., 2006. Anti-rabbit and anti-mouse peroxidase conjugated and ECL detection system (Amersham, IL) were used for detection.

### EXAMPLE 5

### Quantitative RT-PCR

Total RNA was extracted with the RNAeasy kit (Qiagen, Valencia Calif.) following the manufacturer's instructions. 500 ng of RNA were reverse transcribed for 1 hour at 42° C., using MMLV (Clotech, CA) and RNAsin (Promega, WI). cDNA was amplified by Real-Time PCR using the Opticon II (MJ Research). The DYNAMO SYBR green 1 kit (Finnzyme, Finland) was used according to the manufacturer's instructions. Primers were specifically designed between two adjacent exons, using the AutoPrime program. Either custom made or commercial inventoried Taqman probes from Applied Biosystems (Applied Biosystems, CA) were also used, according to the manufacturer's instructions. mRNA levels for each gene were normalized to those of HPRT, using the ΔΔCt method. Primer and probe sequences are available upon request.

### Results and Discussion

### Cdk9-55 is Synthesized Upon the Induction of Muscle Differentiation

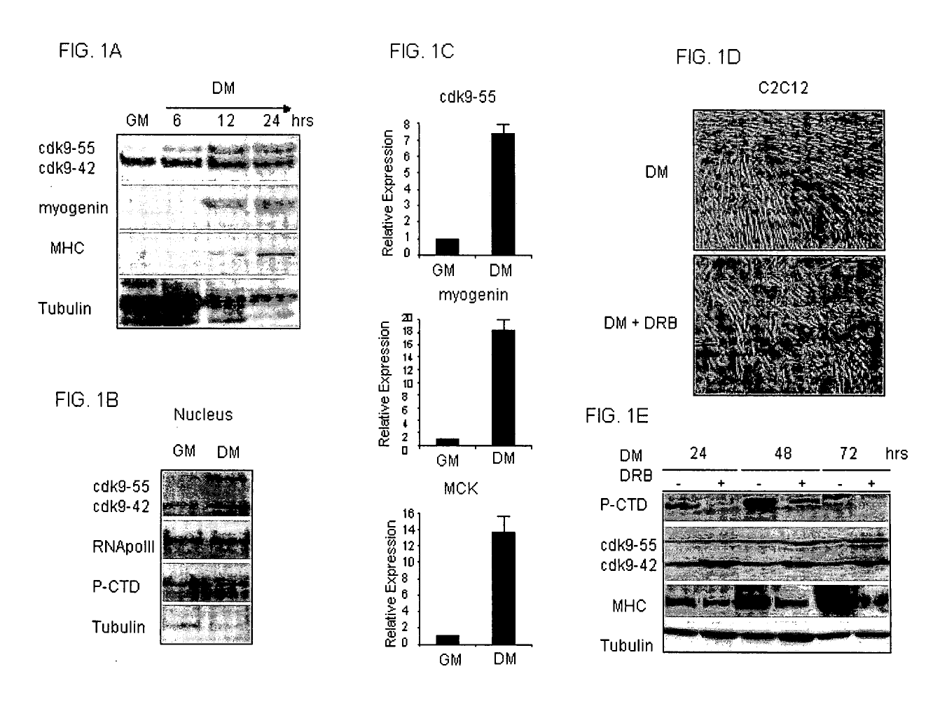

Cdk9-42 protein levels are not affected during the differentiation program, while its kinase activity is clearly augmented and strictly required for MyoD-mediated muscle-specific transcription and myotube formation (Simone et al., Oncogene 21, 4137-4148, 2002; Giacinti et al., J. Cell. Physiol. 206, 807-813, 2006; Simone and Giordano, Cell Death Differ. 14, 192-195. Erratum in: Cell Death Differ. 14, 196, 2007). Cdk9-55 is synthesized upon the induction of muscle differentiation. C2C12 cells either undifferentiated (GM) or induced to differentiate (DM) for the indicated times were analyzed using different techniques. See FIG. 1A-1E. C2C12 mouse myoblasts were cultured and stimulated to terminally differentiate into myotubes. The cdk9-55 isoform was significantly upregulated in cells induced to differentiate, while cdk9-42 displayed similar levels between proliferating and differentiating cells (FIG. 1A). Furthermore, cdk9-55 expression preceded myogenin and MHC expression (FIG. 1A). Nuclear extracts were prepared as described in De Falco et al., Oncogene 21, 7464-7470, 2002. Cdk9-55 localized into the nucleus, and its upregulation coincided with the hyperphosphorylation of the CTD of RNApolII (FIG. 1B). The induction of cdk9-55 expression was confirmed by Real-Time PCR analysis (FIG. 1C). Lastly, cdk9-55 interacted with MyoD (data not shown) as well as cdk9-42 does in C2C12 cells (Simone et al., Oncogene 21, 4137-4148, 2002), indicating that the addition of the 117 N-terminal residues did not alter the conformation of the MyoD-binding region (1-128 aa of cdk9-42) (Simone et al., Oncogene 21, 4137-4148, 2002). Without being limited to a particular theory, the data suggests that this isoform could potentially be the one recruited on the chromatin of muscle-specific genes to activate transcription (Giacinti et al., J. Cell. Physiol. 206, 807-813, 2006).

Since cdk9-55 activity, as well as cdk9-42 activity (Shore et al., Gene 307, 175-182, 2003; Liu and Herman, J. Cell. Physiol. 203, 251-260, 2005), could be inhibited by pharmacological blockade, C2C12 cells were induced to differentiate with the kinase specific inhibitor DRB. C2C12-treated cells failed to undergo terminal differentiation, as confirmed by the presence of mononucleated cells expressing low MHC levels, and the absence of multinucleated myotubes (FIGS. 1D, 1E). This shows novel pharmacological evidence to confirm the essential role of cdk9 function in regulating muscle-specific transcription and myotube formation.

### Cdk9-55 is Induced During Satellite Cell Differentiation

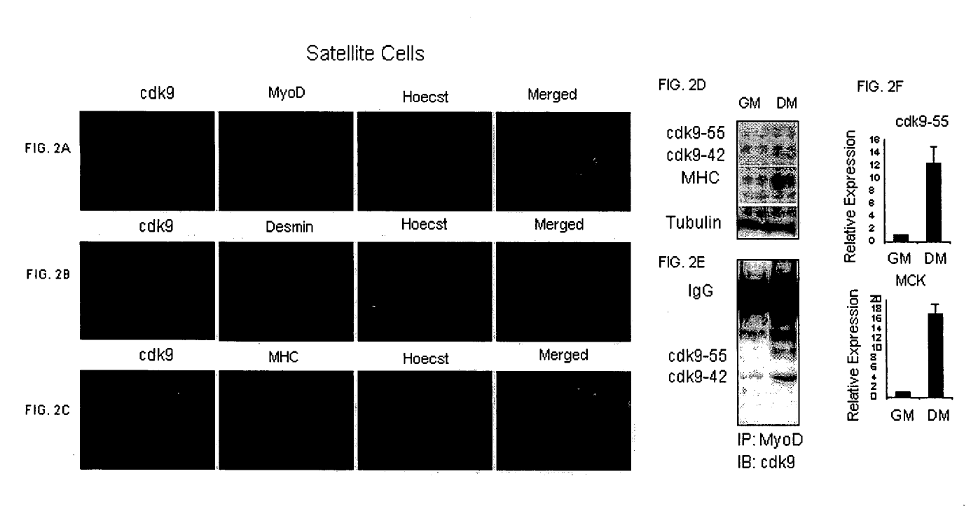

C2C12 cells represent an established cell line originated from mouse satellite cells (Blau et. al, Science 230, 758-766, 1985). To get further insight into the biological role of cdk9-55, a more physiological model using a primary culture of mouse satellite cells obtained by isolating a single muscle fiber (Rosenblatt et al., In Vitro Cell. Dev. Biol. Anim. 31, 773-779, 1995) were employed and then cultured under either proliferating (GM) or differentiating (DM) conditions. In these cells, cdk9 co-localizes with MyoD (FIG. 2A), and its nuclear expression is maintained throughout the satellite cell activation/differentiation process, as shown by the sequential expression of Desmin (FIG. 2B), a cytoplasmic intermediate filament protein involved in myoblast fusion (Smythe et al., Cell Tissue Res. 304, 287-294, 2001), and MHC (FIG. 2C).

At the molecular level, activated satellite cells synthesized detectable levels of cdk9-42 (FIG. 2D), which participate in MyoD complex formation (FIG. 2E). Upon the induction of terminal differentiation, cdk9-42 was up-regulated (FIG. 2D), and its amount in the MyoD complex was enriched (FIG. 2E). MHC is shown as a control for muscle differentiation. Cdk9-55 expression was significantly activated (FIGS. 2D, 2F), and this isoform was recruited by MyoD in differentiating cells (FIG. 2E).

### Cdk9-55 is Induced During Muscle Regeneration In Vivo

Muscle injury was induced in vivo, forcing the muscle to regenerate. Adult C57BL6J mice were subjected to cardiotoxin (CTX) damage in the quadriceps and tibialis muscles (D'Albis et al., Eur. J. Biochem. 174, 103-110, 1988; Musarò et al., Nat. Genet. 27, 195-200, 2001), and the muscle regeneration program was monitored by different techniques.

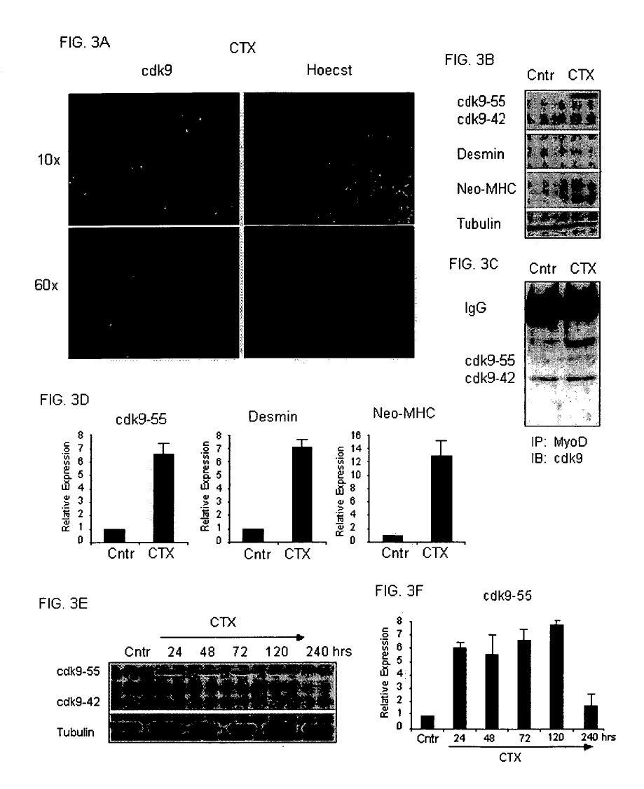

It is noteworthy that 48 hours after CTX damage, cdk9 was highly expressed in the nuclei of regenerating muscles (FIG. 3A) and in newly formed myofibers (data not shown). In fact, the fundamental histological characteristics of the mammalian muscle regeneration process are the focal repair and new formation of small-caliber myofibers with centrally located nuclei (Blayeri et al., Dev. Dyn. 216, 244-256, 1999). At the molecular level, this process was mediated by the up-regulation of Desmin and the expression of the embryonic and neonatal forms of MHC (FIGS. 3B, 3D). Consistently with the data obtained by in vitro studies, we detected a clear induction of cdk9-55 expression in injured skeletal muscles as early as 24 hours and up to 120 hours after injury (FIGS. 3B, 3D, 3E, 3F), while cdk9-42 signals stayed relatively constant over time (FIGS. 3B, 3E). Notably cdk9-55 protein participated in MyoD multiprotein complex in regenerating muscles (FIG. 3C). Interestingly, 240 hours after cardiotoxin (CTX) injection, when the entire process was completed, cdk9-55 expression levels fell abruptly (FIGS. 3E, 3F). This shows that cdk9-55 expression is induced after satellite cell activation and the protein is likely required for differentiation and fusion of myocytes to reconstitute injured myofibers. Once the muscle tissue is repaired cdk9-55 transcription is then switched off.

### Cdk9-55 is Necessary to Complete the Regeneration Process

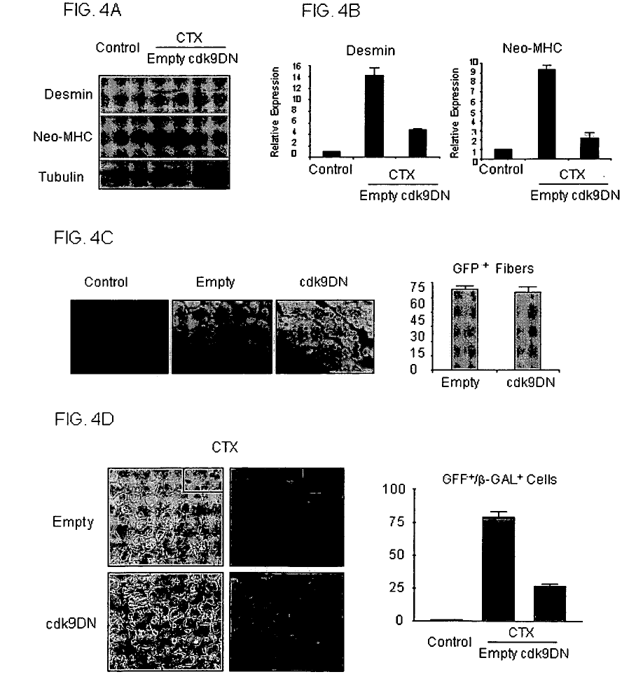

Between day 2 and day 5 post-injury, the regenerative process is at its highest level: myoblasts originating from activated satellite cells exit from the cell cycle and differentiate in myocytes. The dominant negative form of cdk9 (cdk9DN), which is able to strongly inhibit tissue-specific transcription and myotube formation when overexpressed in muscle cells (Simone et al., Oncogene 21, 4137-4148, 2002; Giacinti et al., J. Cell. Physiol. 206, 807-813, 2006; Simone and Giordano, Cell Death Differ. 14, 192-195. Erratum in: Cell Death Differ. 14, 196, 2007) was used to determine whether the inhibition of cdk9-55 activity could affect the completion of muscle regeneration. cdk9DN was electropored in injured muscle and a drastic impairment in muscle regeneration was observed. Immunoblot analysis revealed a significant reduction in the expression of regenerative muscle markers such as Desmin and neonatal MHC (FIG. 4A). Real-Time PCR analysis confirmed that the reduction of their protein levels depended upon a decrease in transcription imposed by cdk9DN (FIG. 4B). To evaluate the transfection efficiency a GFP reporter was employed, and GFP-positive myofibers were counted. (FIG. 4C).

Furthermore, muscle differentiation markers were also downregulated, as demonstrated by the severe inhibition of myogenin expression (data not shown). In order to confirm these important findings injured Desmin-LacZ mice were also electropored and then frozen sections were stained with X-gal (CTX=48 h. These transgenic C57 mice carried a transgene of the Desmin promoter linked to the LacZ reporter gene, which encoded for the beta-galactosidase (β-GAL) enzyme. Interestingly, Desmin promoter is selectively activated in regenerating muscle, making the transgenic mouse a useful model to monitor the different stages of muscle regeneration (Lescaudron et al., Neuromuscul. Disord. 3, 419-422, 1993; Musarò et al., Nat. Genet. 27, 195-200, 2001). Transgenic expression in injured muscles was observed 2 days later, since a blue nuclear product appeared in the presence of the X-gal substrate (Lescaudron et al., Neuromuscul. Disord. 3, 419-422, 1993; Musarò et al., Nat. Genet. 27, 195-200, 2001). A drastic reduction in the number of β-GAL-positive fibers was detected when cdk9DN was overexpressed (FIG. 4D). These results confirmed the requirement of cdk9 kinase activity for adult myogenesis, and highlighted the role of cdk9 in the genome reprogramming necessary to complete the muscle regeneration process in vivo.

### CONCLUSIONS

Cdk9-55 is specifically induced over the course of the regeneration of skeletal muscle and cdk9 kinase activity is essential for C-terminal domain (CTD) hyperphosphorylation and consequent induction of muscle-specific transcription and muscle tissue repair.

All publications, patents and patent applications referred to herein are incorporated herein by reference to the same extent as if each individual publications or patent application was specifically and individually indicated to be incorporated by reference. Although the present invention has been described in terms of specific embodiments, changes and modifications can be made out without departing from the scope of the invention which is intended to be defined only by the scope of the claims.

What is claimed is:

1. A method for regenerating muscle tissue in a patient, comprising:
administering a vector encoding cdk9-55 to the patient in need of muscle tissue regeneration.

2. The method of claim 1, wherein the vector is an adenoviral vector.

3. The method of claim 1, wherein the vector is a retroviral vector.

4. The method of claim 1, wherein the vector is administered intra-arterially.

5. The method of claim 1, wherein the vector is administered intravenously.

6. The method of claim 1, wherein the vector is administered intramuscularly.

7. The method of claim 1, wherein the muscle tissue is smooth muscle.

8. The method of claim 1, wherein the muscle tissue is cardiac muscle.

9. The method of claim 1, wherein the muscle tissue is skeletal muscle.

10. A method for regenerating muscle tissue in a patient, comprising:
administering to the patient a composition comprising a vector capable of expressing cdk9-55 at levels sufficient to promote the regeneration of muscle tissue.

11. A method for regenerating muscle tissue in a patient, comprising:
administering to the patient a composition comprising cdk9-55 proteins.

12. A method for regenerating muscle tissue in a patient, comprising:
co-administering a vector encoding cdk9-55 and Cyt2a to the patient in need of muscle tissue regeneration.

13. A method for regenerating muscle tissue in a patient, comprising:
co-administering a vector encoding cdk9-55 and MyoD to the patient in need of muscle tissue regeneration.

14. A method for regenerating muscle tissue in a patient, comprising:
co-administering a vector encoding cdk9-55 and at least one muscle regulatory factor.

15. A method for regenerating muscle tissue in a patient, comprising:
co-administering a therapeutically effective amount of cdk9-55 protein and at least one muscle regulatory factor.

16. A method for enhancing development of existing muscle tissue comprising:
administering a vector encoding cdk9-55 to a patient in need thereof.

17. A method for enhancing development of existing muscle tissue comprising:
administering to a patient a composition comprising a vector capable of expressing cdk9-55 at levels sufficient to promote the development of existing muscle tissue.

18. A method for enhancing development of existing muscle tissue comprising:
administering to a patient a composition comprising cdk9-55 proteins.

19. A method for enhancing development of existing muscle tissue comprising:
co-administering a vector encoding cdk9-55 and Cyt2a to a patient in need thereof.

20. A method for enhancing development of existing muscle tissue comprising:
co-administering a vector encoding cdk9-55 and MyoD to a patient in need thereof.

21. A method for enhancing development of existing muscle tissue comprising:
co-administering a vector encoding cdk9-55 and at least one muscle regulatory factor.

22. A method for enhancing development of existing muscle tissue comprising:
co-administering a therapeutically effective amount of cdk9-55 protein and at least one muscle regulatory factor.
